(19) United States
(12) Patent Application Publication
(10) Pub. No.: US 2022/0280220 A1
(43) Pub. Date: Sep. 8, 2022

(54) DEVICE FOR AN ELECTROSURGICAL INSTRUMENT

(71) Applicants: Maslanka Patentverwaltung GmbH, Freiburg (DE); KLS MARTIN GMBH + CO. KG, Freiburg (DE)

(72) Inventors: Herbert MASLANKA, Tuttlingen (DE); Bernhard HUG, Freiburg (DE)

(73) Assignees: Maslanka Patentverwaltung GmbH, Freiburg (DE); KLS MARTIN GMBH + CO. KG, Freiburg (DE)

(21) Appl. No.: 17/637,735

(22) PCT Filed: Aug. 17, 2020
(86) PCT No.: PCT/EP2020/072949

§ 371 Date: Feb. 23, 2022

(30) Foreign Application Priority Data
Aug. 26, 2019 (DE) 10 2019 212 780.0

(51) Int. Cl. A61B 18/04; A61B 90/92; A61B 90/96; A61B 90/98

(52) U.S. Cl. CPC A61B 18/042; A61B 90/92; A61B 90/96; A61B 90/98; A61B 2018/00589

(57) ABSTRACT

The invention relates to a device for an electrosurgical instrument, comprising a coupling unit, said coupling unit having a fluid line connection and a current line connection which are designed to carry fluid and current, via a fluid line of the device and at least one current line of the device, between a higher-level assembly and a distal end of the device and a filter, the filter being provided separately from the coupling unit in a fluid flow path of the fluid line, such that the fluid flowing through the fluid line flows through the filter.

The invention relates to a device for an electrosurgical instrument.

Electrosurgical instruments are used in particular in endoscopy, for example for plasma coagulation. In this case, a tubular device is connected to a gas source and a high-frequency generator, and gas is then conducted to the distal end of the tubular device, where it is converted into a plasma with the aid of electric current from the high-frequency generator. However, a backflow from the distal end of the tubular device to the gas source or the high-frequency generator can result in contamination.

For this purpose, WO 2004/002345 A1 proposes integrating a filter into a plug connector of the tubular device for connection to the high-frequency generator or the gas source. As a result, however, a corresponding plug connector is enlarged in such a way that its handling is severely impaired and it may also be necessary to use a high-frequency generator or a special gas source specially designed for the plug connector, since the plug connector cannot be used with standard devices.

It is therefore the object of the present invention to provide a device for an electrosurgical instrument which is easier to handle.

This object is achieved according to the invention by a device for an electrosurgical instrument, comprising a coupling unit for coupling the device to a higher-level assembly not belonging to the device, said coupling unit having at least one fluid line connection and at least one current line connection which are designed to carry fluid and current, via at least one fluid line of the device and at least one current line of the device, between the higher-level assembly and a distal end of the device when the coupling unit is coupled to the higher-level assembly, and a filter which is designed to absorb particles of a predetermined minimum size in order to separate them from the fluid, the filter being provided separately from the coupling unit in a fluid flow path of the fluid line, such that the fluid flowing through the fluid line flows through the filter.

This arrangement makes it possible to use conventional standard coupling units, for example plug connectors, on the device according to the invention, so that the device according to the invention can be used with existing standard equipment.

Furthermore, providing the filter separately from the coupling unit may allow the filter to be selected in relation to specifications required for a particular application, such as pore size, flow rate, etc., rather than having to select the filter in relation to a limited space available. In particular, filters in common standard formats can also be used, for example as circular or rectangular filters.

If the filter is provided separately from the coupling unit, it is possible to form the working length of the device from the filter to the distal end with high-quality tube material and to form the supply line length from the coupling unit to the filter with a different, in particular cheaper, material.

In a further development of the present invention, the filter can be provided in a range from 1 cm to 50 cm, in particular in a range from 5 cm to 30 cm, more preferably in a range from 10 cm to 20 cm, adjacent to the coupling unit as measured along the fluid flow path of the fluid line. Providing the filter in such a region of the coupling unit can have the advantage that the filter is also provided close to the higher-level assembly, such as the high-frequency generator and/or the fluid source, which can prevent the filter from resting on the floor of an operating theatre and being soiled or damaged by a person stepping on the filter or rolling a chair over it. On the other hand, this can prevent the filter from disrupting handling of the device in a working environment of the device, for example a surgical environment.

In particular, the filter can be surrounded by a casing which fluidly seals the fluid flow path of the fluid line through the filter to the outside. The filter can be removable from the casing and thus exchangeable and/or the filter can be removed from the device together with the casing and thus exchangeable.

Advantageously, with respect to the filter provided in the casing, the current line can run outside the casing. This means that at the latest at a point at which the fluid line leads into the casing of the filter, a branching device can be provided, at which the current line branches off from the fluid line, then runs separately around the casing of the filter and can then be reunited with the fluid line. Although it can generally be conceivable to also route the current line through the filter, however, by redirecting the current line around the outside of the casing of the filter, simplified filters which are thus reduced in terms of costs and/or installation space can be used.

In this case, the casing surrounding the filter and the current line running around the casing can be surrounded by a housing which comprises an electrically insulating material. In this way, the filter and the current line can be protected from being damaged by external influences. Furthermore, an operator can be prevented from coming into contact with the current line should it be routed as an uninsulated conductor around the casing of the filter.

The filter can be designed to absorb particles of a predetermined minimum size from 0.1 μm, in particular from 0.2 μm. The smaller the minimum size of particles that are intercepted by the filter, the better contaminants, in particular germs, for example viruses and/or bacteria, can be filtered out of the fluid. However, with a finer filter, the flow rate decreases (at the same pressure). In order to allow the use of a fine filter, such as a filter that filters out particles from 0.2 μm, without significantly changing the flow properties of the fluid through the device, an absorbing surface of the filter must be increased in particular, since an increase in the pressure used can lead to undesirable effects in the flow properties of the fluid (see below). Since the device according to the invention does not limit the installation space for the filter, for example when the filter is provided in the coupling unit, the filter surface can be increased accordingly in order to be able to use such a fine filter without significantly changing the flow properties of the fluid.

Filters that are designed to filter particles from a size of 0.4 μm or even 0.45 μm cannot generally be called sterile filters because their filter properties are not sufficient to achieve sterility.

Furthermore, the filter can comprise a hydrophobic or hydrophobically coated material. It can thus be prevented that moisture present in the fluid can be absorbed by the filter material, as a result of which the filter can swell, for example, and its filter properties can change.

In a particular embodiment of the present invention, the fluid can comprise gas, in particular argon. Use of argon for surgical applications such as coagulation is well known and accepted. In particular, the gas emitted via the distal end of the device can be converted into a plasma by the current provided via the current conductor.

The fluid line connection, the fluid line and/or the filter can be adapted, in particular at a pressure of at most 2 bar, to allow a fluid flow rate of 0.1 l/min to 12 l/min, in particular from 0.4 l/min to 1.2 l/min. If the flow velocity exiting the distal end of the device is too great, turbulence can occur in the fluid flow exiting through the distal end of the device, which can mix this fluid with fluid present in the vicinity of the distal end, so that plasma generated by the fluid flow can break off. If the flow velocity exiting the distal end of the device is too low, no laminar fluid flow can be generated from the distal end of the device, as a result of which the use of the device, particularly in a surgical environment, can be disrupted or even prevented.

The fluid line and the current line can also be routed in a common line, in particular a hose line which comprises PTFE. In particular, this can have the advantage that a tube in which both the fluid line and the current line are routed can have a smaller outer diameter than the sum of separately running current and fluid lines. Inserting the device into a surgical device, such as an endoscope, can also be simplified as a result. In general, however, it is also conceivable for the fluid line and the current line to be routed separately.

In particular, the filter can be substantially circular. A circular design of the filter can have an advantageous effect on the flow properties of the fluid through the filter, for example because there can be no turbulence or “dead spaces” in corners of the filter. A circular design of the filter can also have an advantageous effect on the installation space required and the rotational symmetry of the device.

Furthermore, the filter can have a cross-sectional area through which fluid flows in the range from 100 mm² to 10000 mm², in particular from 500 mm² to 1000 mm², more preferably from approximately 700 mm². The cross-sectional area can in particular be measured orthogonally to a main flow direction of the fluid through the filter. When using conventional filter materials which are web-like, the measured cross-sectional area can correspond to a planar extension of the main surface of the filter material.

Advantageously, the device can comprise a machine-readable identifier which is associated with a type of device, in particular a length from the coupling unit to the distal end or a diameter of the device and/or a diameter of the fluid line and/or a fluid flow rate defined by the device. This identifier can, for example, be suitable for being read by a corresponding reading unit, which is arranged in the higher-level assembly, such as the high-frequency generator and/or the fluid source. In this way, the identifier can in particular be read automatically when the device is coupled to the higher-level assembly. It is conceivable that the device type specifications assigned to the identifier can be taken directly from the identifier and/or that these specifications can be taken from a database connected to the reading unit, in which an assignment of predetermined specifications to a respective identifier is stored.

For this purpose, the identifier can be provided on the coupling unit. In this way, the identifier can be provided close to the higher-level assembly when it is coupled to the higher-level assembly. Furthermore, the coupling unit can be designed to be able to be coupled to the higher-level assembly in precisely one orientation, so that in this case an identifier provided on such a coupling unit, when coupled to the higher-level assembly, also always assumes the same relative orientation to the higher-level assembly. Accordingly, the reading unit can be designed to read the identifier at a predetermined point at which the coupling unit is coupled to the higher-level assembly.

The identifier can be of the RFID type and/or a barcode and/or a two-dimensional code and/or a colour code. Of course, in general, the identifier can be of any type that lends itself to being read by the reading unit in order to distinguish a first device, for example of a predetermined length, from a second device of a different length. In particular for use of the device in a surgical environment, a non-optical method for identifying the identifier can be used, such as RFID, since the RFID method works largely independently of whether the coupling unit, and thus the identifier, is soiled or is covered.

The present invention will be described in greater detail below by means of an embodiment with reference to the accompanying drawings. In the drawings:

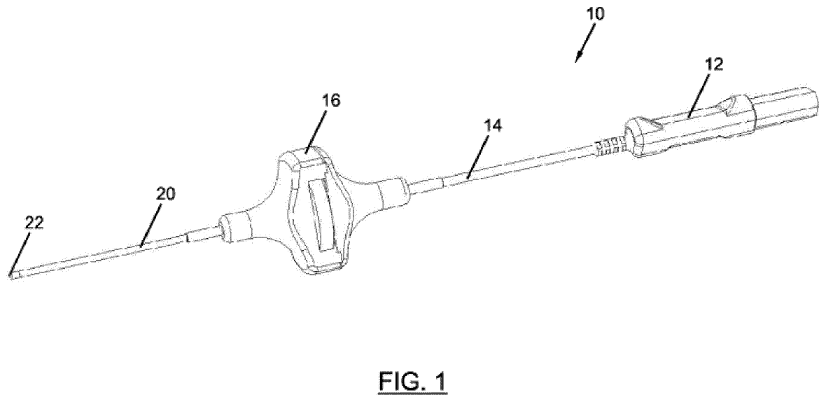

FIG. 1 is a perspective view of an embodiment of a device according to the invention;

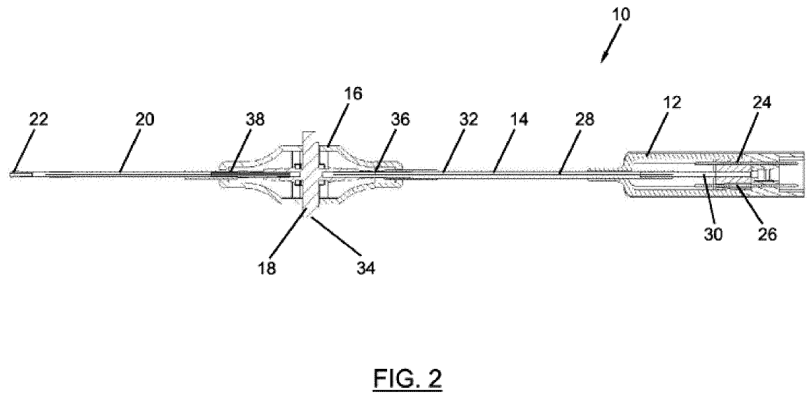

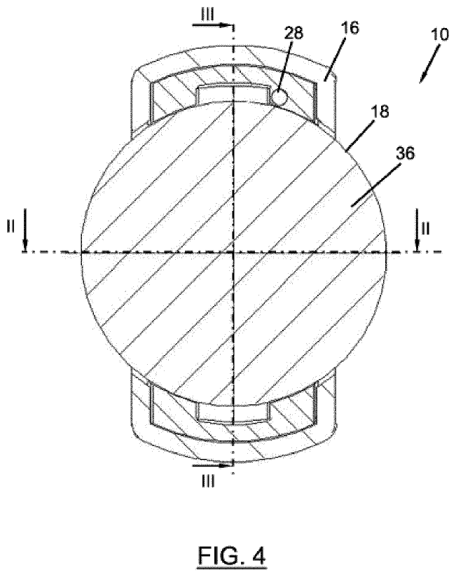

FIG. 2 is a side cross-sectional view of the device from FIG. 1 taken along line II-II from FIG. 4;

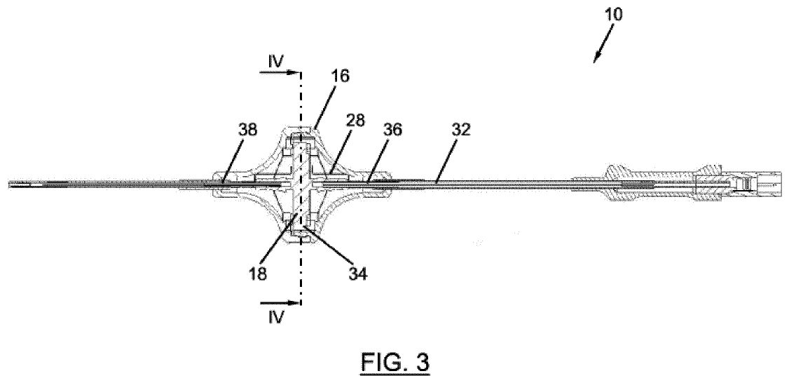

FIG. 3 is a further side cross-sectional view of the device from FIG. 1 taken along line III-III from FIG. 4; and

FIG. 4 is a cross-sectional view of the device from FIG. 1 taken along line IV-IV from FIG. 2.

In FIG. 1, a device according to the invention is generally denoted with reference sign 10. The device 10 comprises a coupling unit 12 which is designed to be coupled to a higher-level assembly (not shown). A first tube 14 is connected to the coupling unit 12 and a housing 16 is provided at the end of the tube opposite the coupling unit 12. A filter 18 is placed in the housing 16 and protrudes laterally from the housing 16. In this way, the housing 16 can be designed to be weight-optimised. In a further embodiment, the housing 16 can of course also be closed, designed to surround the filter. At the end of the housing 16 opposite the first tube 14, the housing 16 is connected to a second tube 20, which is represented in a greatly shortened form in the embodiment shown. The second tube 20 ends in a distal end 22.

With reference to FIG. 2, it can be seen that the coupling unit 12 has two current line connections 24, 26, which run into the first tube 14 and run there as current conductors 28 to the distal end 22 of the device 10, via which current can be carried from the higher-level assembly, in particular a high-frequency generator, to the distal end 22. Furthermore, the coupling unit 12 has a fluid line connection 30, which also runs into the first tube 14 and runs there as a fluid line 32 to the distal end 22 of the device 10, via which fluid can be carried from the higher-level assembly, in particular a fluid source, to the distal end 22.

The filter 18 is provided in a flow path, which is defined by the fluid line 32, in such a way that all of the fluid flowing through the fluid line 32 also flows through the filter 18. In order to fluidly seal the filter 18 from the outside, the filter 18 is surrounded by a casing 34.

In order to be able to use a filter 18 which comprises a flat filter material not having any openings that are significantly larger than a predetermined pore size of the filter (see FIG. 4), the filter 18 is connected to the first tube 14 or to the second tube 20 via conduits 36, 38 made of an electrically conductive material, for example stainless steel, and the current conductor 28 branches off from the fluid line 32 at the conduits 36, 38 and runs in the housing 16 around the filter 18 or its casing 34.

It can also be seen in FIG. 4 how the current conductor 28 is routed around the filter 18 in the housing 16. With reference to FIG. 4, it can additionally be seen that the filter 18 and its casing 34 are substantially circular.

1. Device for an electrosurgical instrument, comprising
a coupling unit for coupling the device to a higher-level assembly not belonging to the device, said coupling unit having at least one fluid line connection and at least one current line connection which are designed to carry fluid and current, via at least one fluid line of the device and at least one current line of the device, between the higher-level assembly and a distal end of the device when the coupling unit is coupled to the higher-level assembly, and
a filter which is designed to absorb particles of a predetermined minimum size in order to separate them from the fluid, the filter being provided separately from the coupling unit in a fluid flow path of the fluid line, such that the fluid flowing through the fluid line flows through the filter,
wherein the device further comprises a tube connected to the coupling unit,
wherein a housing is provided at the end of the tube opposite the coupling unit, and
wherein the filter is placed in the housing and protrudes from the housing.

2. Device according to claim 1,
wherein the filter is provided in a range from 1 cm to 50 cm adjacent to the coupling unit as measured along the fluid flow path of the fluid line.

3. Device according to either claim 1,
wherein the filter is surrounded by a casing which fluidly seals the fluid flow path of the fluid line through the filter to the outside.

4. Device according to claim 3,
wherein the current line runs outside the casing with respect to the filter provided within the casing.

5. Device according to claim 4,
wherein the casing surrounding the filter and the current line running around the casing are surrounded by the housing, which comprises an electrically insulating material.

6. Device according to claim 1,
wherein the filter is designed to absorb particles of a predetermined minimum size from 0.1 μm.

7. Device according to claim 1,
wherein the filter comprises a hydrophobic or hydrophobically coated material.

8. Device according to claim 1,
characterised in that the fluid comprises gas.

9. Device according to claim 1,
wherein the fluid line connection, the fluid line and/or the filter are adapted, at a pressure of at most 2 bar, to allow a fluid flow rate of 0.1 l/min to 12 l/min.

10. Device according to claim 1,
wherein the fluid line and the current line are routed in a common line.

11. Device according to claim 1,
wherein the filter is substantially circular.

12. Device according to claim 1,
wherein the filter has a cross-sectional area through which fluid flows in the range from 100 mm² to 10000 mm².

13. Device according to claim 1,
wherein the device comprises a machine-readable identifier which is associated with a length from the coupling unit to the distal end or a diameter of the device and/or a diameter of the fluid line and/or a fluid flow rate defined by the device.

14. Device according to claim 13,
wherein the identifier is provided on the coupling unit.

15. Device according to claim 1,
wherein the identifier is of the RFID type and/or a barcode and/or a two-dimensional code and/or a colour code.

16. Device according to claim 2, wherein the filter is provided in the range of 5 cm to 30 cm adjacent to the coupling unit as measured along the fluid flow path of the fluid line.

17. Device according to claim 16, wherein the filter is provided in the range of 10 cm to 20 cm adjacent to the coupling unit as measured along the fluid flow path of the fluid line.

18. Device according to claim 9, wherein the fluid line connection, the fluid line and/or the filter are adapted to allow a fluid flow rate of 0.4 l/min to 1.2 l/min.

19. Device according to claim 10, wherein the hose line comprises PTFE.

20. Device according to claim 13, wherein the filter has a cross-sectional area through which fluid flows in the range from 500 mm² to 1000 mm².

21. Device according to claim 20, wherein the filter has a cross-sectional area through which fluid flows in the range from approximately 700 mm².
